(19) United States
(12) Patent Application Publication
(10) Pub. No.: US 2022/0399103 A1
(43) Pub. Date: Dec. 15, 2022

(54) Method and process for amassing time increments of procedure steps to determine perioperative surgery duration estimates.

(71) Applicants: Martin A. Martino, MD, Ocean, NJ (US); Anthony J. Salvaggio, Allentown, PA (US); Charles S. Anderson, Harrisburg, PA (US); Eric R. Edgar, Hellertown, PA (US)

(72) Inventors: Martin A. Martino, MD, Ocean, NJ (US); Anthony J. Salvaggio, Allentown, PA (US); Charles S. Anderson, Harrisburg, PA (US); Eric R. Edgar, Hellertown, PA (US)

(21) Appl. No.: 17/347,552

(22) Filed: Jun. 14, 2021

(51) Int. Cl. G16H 40/20; G16H 40/67; G16H 20/40; G16H 50/70; G16H 50/20; G16H 10/60; G16H 30/20; A61B 90/00; A61B 34/00

(52) U.S. Cl. CPC G16H 40/20; G16H 40/67; G16H 20/40; G16H 50/70; G16H 50/20; G16H 10/60; G16H 30/20; A61B 90/37; A61B 34/25; A61B 2034/252

(57) ABSTRACT

Systems and methods for monitoring medical perioperative time periods by surgical and non-surgical procedure steps. Particular embodiments relate to capturing and accumulating medical intraoperative procedure step durations. A method for valuing a surgery timeline may be based on surgeon(s), surgical team, CPT codes, patient comorbidities and other factors. The method may receive historical metrics from prior perioperative steps and intakes the surgeon's duration estimate or the organization administrative staffs duration estimate or both. The method displays the surgery timeline by applying the historical prior surgery metrics with input from the facility user.

The facility provides transparency into how all the perioperative step's times are accounted for within the period of time extending from when the patient enters the organization's facility (e.g. hospital, clinic, doctor's office, and the like) for surgical procedure until the time the patient is discharged.

These various combinations are desirable to provide information to the surgeon, surgical team and organization's administrative staff to improve case efficiency, patient care and user satisfaction.

### CROSS-REFERENCE TO RELATED APPLICATIONS

Not Applicable.

### FEDERALLY SPONSORED RESEARCH

Not Applicable.

### SEQUENCE LISTING OR PROGRAM

Submitted as a separate file named SurgicalTrackerSourceCode.pdf.

### INFORMATION DISCLOSURE STATEMENT

No prior art is cited.

### REFERENCES CITED [REFERENCED BY]

Search using conditions: (((time AND increments) AND operating) AND room) returned 24,637 results in the United States Patent and Trade Office databases on Jun. 1, 2021. Review of these patents resulted in zero patents applicable to the claims of this patent.

The following patents are referenced:

| Patent Number | Patent Date | Inventor |
|---|---|---|
| 2002/0055918 | May 2002 | Hlathein |
| 2003/0204411 | October 2003 | Beyersdorf |
| 2004/0044546 | March 2004 | Moore |
| 2004/0064342 | April 2004 | Browne et al. |
| 2004/0169673 | September 2004 | Crampe |
| 2005/0015276 | January 2005 | Sullivan |
| 2006/0142739 | June 2006 | DiSilestro |
| 2007/0078678 | April 2007 | DiSilvestro |
| 2007/0136097 | June 2007 | Demers et al. |
| 2007/0136218 | June 2007 | Bauer |
| 2007/0282195 | December 2007 | Masini |
| 2008/0246610 | October 2008 | Packert et al. |
| 2008/0281636 | November 2008 | Jung et al. |
| 2009/0089081 | April 2009 | Haddad |
| 2010/0305970 | December 2010 | McLaren et al. |
| 2010/0305971 | December 2010 | McLaren et al. |
| 2010/0324933 | December 2010 | Giap |
| 2011/0137680 | June 2011 | Sweeney |
| 7,896,869 | Mar. 1, 2011 | DiSilvestro, et al. |
| 2012/0101841 | April 2012 | Kobayashi |
| 2012/0239420 | September 2012 | Stapelfeldt et al. |
| 2012/0323597 | December 2012 | Woolford |
| 2013/0211421 | August 2013 | Abovitz |
| 2013/0285947 | October 2013 | Hunter et al. |
| 2016/0018963 | January 2016 | Robbins et al. |
| 10489023 | Nov. 2019 | Robbins et al. |
| 10,943,454 | Mar. 9, 2021 | Shelton, IV, et al. |
| 10,467,553 | Nov. 5, 2019 | Blecharczyk et al. |
| 10,997,963 | May 4, 2021 | Vikas Baligar, et al. |

### OTHER REFERENCES

Primary Examiner: N/A

Attorney, Agent or Firm: N/A

### TECHNICAL FIELD

This invention relates to the field of healthcare medical safety and quality systems and methods within perioperative surgery which is from the time the patient goes into the organization (e.g. hospital, clinic, doctor's office and the like) for surgery until the time the patient is discharged. In particular, it relates to predicting the case duration with greater accuracy using previous case step duration history. The predicted case durations are compared to independent case predictions from surgeons and operating teams. In addition certain steps related to safety checklists enhance the safety of surgical procedures for the patient.

### BACKGROUND ART

Surgery duration estimates are one of the key metrics required for Operating Room (OR) preparation and scheduling. Historically perioperative surgery duration estimates have been communicated verbally or hand written from the surgeon, the organization's OR administration staff or both. Individual surgeon's methods for estimating perioperative surgery duration varies widely. In later embodiments the communication was manually keyed into a terminal device with subsequent embodiments including simple averaging (arithmetic average) method of a small sample of prior cases for predicting the next scheduled perioperative surgery duration without regard of the prior surgical procedure(s) performed compared to the surgical procedure(s) to be performed or other metrics that influence actual surgery case durations.

A limitation of the current field is in using the simple average (arithmetic average) method to determine perioperative surgery duration. Simple average method uses equal weighting to each number (duration time) in the calculation. Simple average does not take into account factor values in relation to their relative impact on the surgery duration. Therefore what is clearly needed is an algorithm with real-time perioperative transparent method for improving the accuracy of surgery case estimations.

Another limitation may be that if an intended perioperative step is omitted or performed out of order, and the surgical outcome is influenced, there is no way, absent accurate records, to evaluate the situation.

### DISCLOSURE OF THE INVENTION

Initial development of this embodiment began with the surgeon using a written schedule in the OR and receiving verbal agreement concerning surgical team metrics with the surgical team. Once this embodiment was proven in written form, translation to a smart digital platform was used intra-operatively and the inclusion of voice action, digital display, and an algorithm that included three case duration estimates for surgical case comparison. This embodiment lessens surgery case time variability with more predictable scheduling of cases, enhanced perioperative efficiency, surgical team performance and improved patient safety.

Some current prior art captures limited case step durations by a user entering the information into a terminal, captures the case step duration data during the operation, or captures the case step duration post-perioperative or a combination of the aforementioned. These methods may result in cumbersome data capture, inaccuracies and lack of real time feedback to the surgeon, surgical team and organization's administration.

Some current prior art captures data on paper which may be manually entered into a system at the end of the day. This method results in data error entry, bias and lack of informative data to improve perioperative process flow in real-time.

### SUMMARY OF THE INVENTION

Shortcomings of prior operating room management systems are addressed by the systems and methods presented herein. This embodiment includes the methods and processes for amassing time increments from organization's operating room (OR) administration, surgeon and machine learning predictive model or any other artificial intelligence mathematical algorithm for case duration estimates and monitoring actual durations for the perioperative surgery process. This invention has added new metrics to the prior art which are presented in the Detailed Description section.

Some embodiments include a processor-driven method of providing perioperative schedule durations to a web-responsive designed user interface for supporting terminal, voice assistant, and the like devices, the method comprising operating one or more processors to execute stored program instructions causing the one or more processors to receive a notification that a trigger event has occurred, the notification comprising a case event identifier, a surgeon identifier, a surgery procedure identifier and one or more case event data items, provides an estimate of the surgery duration obtained from a repository based on at least one of: the surgery event identifier, the surgeon identifier and at least one of the one or more event data items, create a perioperative schedule duration comprising case information based on the case identifier, the surgeon identifier, surgery procedure identifier and at least one of the one or more event data items, and display the predicted perioperative duration on a terminal device. The embodiment uses the predicted perioperative duration to track and display the specific predicted perioperative case step durations with the actual perioperative case step durations throughout the perioperative event. The current embodiment utilizes a machine learning predictive model, and/or any other artificial intelligence technique, herein called an estimation algorithm, to determine the perioperative step duration estimates.

An embodiment may display three (3) or more patient safety checklists digitally and presents to the operative team uniformly for completion, causing the one or more processors to receive a notification that a trigger event has occurred, the notification comprising a surgery event identifier and a checklist identifier capturing the datetime the checklist was presented and acknowledged.

Some embodiments are augmented with voice commands to the processor-driven method or allows for traditional direct entry to a terminal device. This or other embodiments exchanges surgeon, patient and surgery data with an organization's Electronic Medical Record (EMR) system using Health Level Seven (HL7) standard transactions.

The features and advantages described in this summary and the following detailed description are not all-inclusive. Many additional features and advantages will be apparent to one of ordinary skill in the art in view of the drawings, specification, and claims.

The terms and phrases as indicated in parenthesis (“ ”) in this section are intended to have the meaning ascribed to them in this section applied to them throughout this document including the claims unless clearly indicated otherwise in context.

The term “or” as used in this specification and the appended claims is not meant to be exclusive rather the term is inclusive meaning “either or both”.

References in the specification to “one embodiment”, “an embodiment”, “a preferred embodiment”, “an alternative embodiment”, “embodiments”, “variations”, “a variation” and similar phrases mean that a particular feature, structure, or characteristic described in connection with the embodiment(s) or variation(s) is included in at least an embodiment or variation of the invention. The appearances of the phrase “in one embodiment” or “in one variation” in various places in the specification are not necessarily all referring to the same embodiment or variation.

### BRIEF DESCRIPTION OF THE DRAWINGS

The detailed description is described with reference to the accompanying figures. In the figures, the left-most digit(s) of a reference number identifies the figure in which the reference number first appears. The same reference numbers in different figures indicate similar or identical items. FIG. 1 and the other figures use like reference numerals to identify like elements. A letter after a reference numeral, such as “1013A” indicates that the text refers specifically to the element having that particular reference numeral. A reference numeral in the text without a following letter, such as “1103” refers to any or all of the elements in the figures bearing that reference numeral (e.g. “1103” in the text refers to reference numerals “1103A” and/or “1103B” in the figures).

For simplicity of illustration, network connections, standard process such as user authentication, etcetera. are not depicted as they are known to those with skill in the art. When they are shown, it is purely for illustrative purposes and not intended to capture all embodiments of the invention disclosed.

FIG. 1 is a diagram of a computer-implemented interactive surgical tracking system, in accordance with the preferred aspect of the present disclosure.

FIG. 2 is a block diagram illustrating the exemplary data stores and modules inside the surgical tracker system architecture, in accordance with an embodiment of the invention.

FIG. 3 is a flowchart of an exemplary method for creating surgery case information for the surgical tracking system, in accordance with one embodiment of the invention.

FIG. 4 is an exemplary user interface for surgical tracking system to add a surgery case in accordance with one embodiment of the invention.

FIG. 5 is an exemplary user interface for a surgical tracking system being used to monitor a surgical procedure in an operating room and immediately display the metrics, in accordance with one embodiment of the invention.

FIG. 6 is an exemplary database diagram of the persistent data stores used in accordance with one embodiment of the invention.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE INVENTION

The various embodiments and variations thereof illustrated in the accompanying figures and/or described herein are merely exemplary and are not meant to limit the scope of the invention. It is to be appreciated that numerous variations of the invention have been contemplated as would be obvious to one of ordinary skill in the art with the benefit of this disclosure. Rather, the scope and breadth afforded this document should only be limited by the claims provided herein while applying either the plain meaning to each of the terms and phrases in the claims or the meaning clearly and unambiguously provided in this specification.

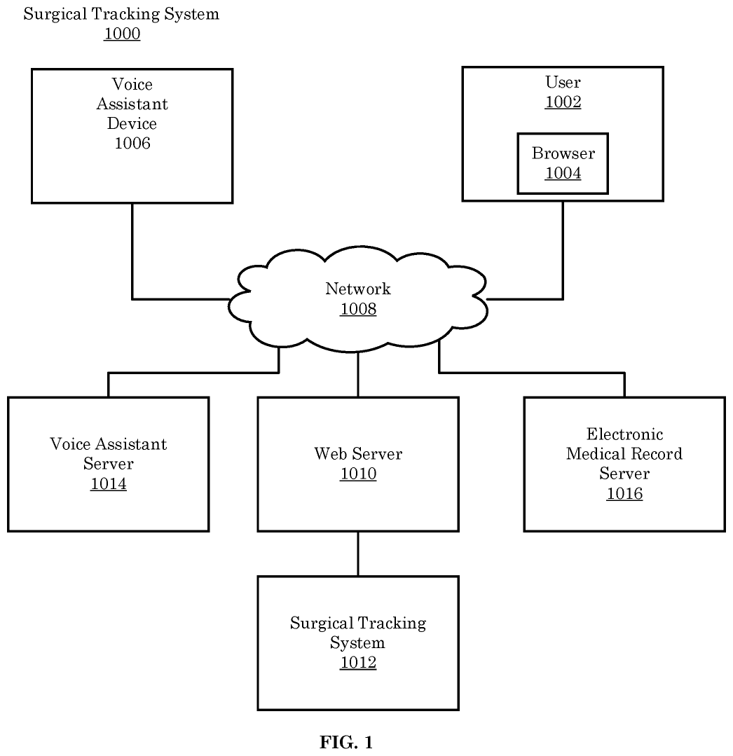

Referring now to FIG. 1, there is shown the surgical tracking system 1000 as adapted to support one embodiment of the present invention. The network 1008 represents the communication pathways between the surgical tracking system user 1002, the voice assistance device 1006 and the surgical tracking system 1012. In one embodiment, the network is the Internet. The network may also utilize dedicated or private communication links (e.g. WAN, MAN, or LAN) that are not necessarily part of the Internet. The network uses standard communications technologies and/or protocols.

The web server 1010 presents user interface in the form of web page or other web content, which form the basic interface to the user 1002 and browser 1004. The user employs the respective client device browser 1004 to access one or more user interfaces, and provide data to the surgical tracking system 1012. In the context of this application, “data” is understood to include information about a perioperative case, a patient information, surgeon(s) performing the case and the like. For example, for information related to a surgery, the data can include information such as one or more current procedural terminology (CPT) codes, one or more International Classification of Diseases (ICD)s codes, an operating room designation, a surgery scheduled start date, a surgery scheduled start time, a patient's body mass index, a patient's height, a patient's weight and the like.

The voice assistant device 1006 may receive input from users, 1002 by receiving spoken commands, which are converted to signals by the voice assistant device 1006 and/or by a cloud service, and then processed, such as by an exchange of data with the voice assistant service, 1014.

The electronic medical record service 1016 exchanges in a bi-directional manner surgeon, patient surgery data and the like between the surgical tracking system 1012 and an organization's EMR using Health Level Seven (HL7) standard transactions or other data exchange transaction format.

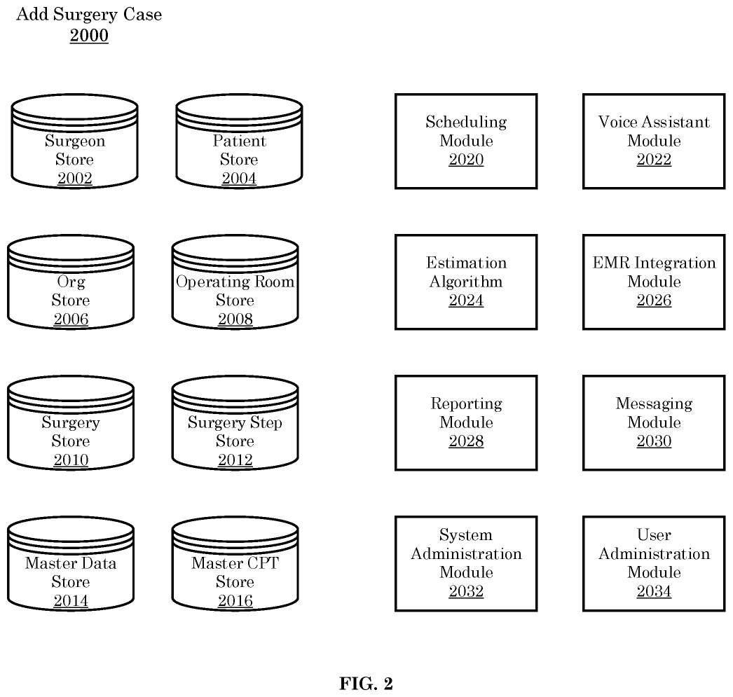

Referring to FIG. 2, in one embodiment the surgical tracking system architecture 2000 comprises a surgeon store 2002, a patient store 2004, an org (organization) store 2006, an operating room store 2008, a surgery store 2010, a surgery step store 2012, a master data store 2014, a master CPT store 2016, a scheduling module 2020, a voice assistant module 2022, an estimation algorithm module 2024, an EMR integration module 2026, a messaging module 2030, a system administration module 2032, and a user administration module 2032. Those artisans skilled in the art will appreciate that the surgical tracking system architecture 2000 may contain other modules that are not described herein. In addition, conventional elements, such as firewalls, authentication systems, security tools, network management tools, load balancers, and so forth are not shown as they are not material to the invention.

The surgical tracking system architecture 2000 may be implemented using a single computer, or a network of computers, including cloud-based computer implementations. The computers are preferably server class computers including one or more high performance CPUs and 1 GB or more of main memory and running an operating system such as LINUX or variants thereof. The operations of the surgical tracking system architecture 2000 as described herein can be controlled through either hardware or through computer programs installed in non-transitory computer storage and executed by the processors to perform the functions described herein. The various stores (e.g., surgeon store 2002, patient store 2004, etcetera.) are implemented using non-transitory computer readable storage devices, and suitable database management systems for data access and retrieval. The surgical tracking system 1000 includes other hardware elements necessary for the operations described here, including network interfaces and protocols, web responsive input and out devices for data entry display, printing, or other presentations of data.

The surgeon store 2002 persistently stores data describing users that can perform surgeries (i.e., surgeons) in the surgical tracking system 2000, and is one means for performing this function. Each surgeon is represented by surgeon object 3002, which may also be called a user. Information about a surgeon include surgeon personal information such as a surgeon unique identifier, name, email address, phone number, pager, preferences, CPTs the surgeon performs and the like.

The patient store 2004 persistently stores data describing patients that exist in the surgical tracking system 2000. Each patient is represented by a patient object. Patient store 2002, information includes data such as patient unique identifier, Body Mass Index (BMI), American Society of Anesthesiologists (ASA) score, International Classification of Diseases (ICD-10) code(s), one or more patient comorbidities, basic personal information, and the like.

The org store 2006 persistently stores data describing orgs that separate data in the surgical tracking system 2000. Every patient, surgeon, operating room belongs to one or more organizations. An example of an organization could be a medical practitioner office, surgery center, hospital, or a network of hospitals.

The operating room store 2008 persistently stores data describing operating rooms that exist in the org 2006 as part of the surgical tracking system 1000. A surgery 2010 exists inside an operating room with a patient 2004 and a surgeon 2002 assigned to it for a period of time on a specified date.

The surgery store 2010 persistently stores data describing surgeries that is a combination of a surgeon 2002 performing a surgery on a patient 2004 in an operating room 2008 under an org 2006 inside the surgical tracking system 2000 in this embodiment.

The surgery step store 2012 persistently stores data describing surgery steps that exist for a specific surgery 2010 in the surgical tracking system 2000. Surgery steps are determined and sequenced by the surgery 2010 that is being performed. A surgery step may be optional, required, or required at some point, but may be performed out of sequence. The surgery step 2012 may have step duration estimates provided by the surgeon, the organization, the estimation algorithm or any combination thereof.

During the surgery the surgery step may be updated as completed at a specific time and recorded as a timestamp inside the surgery 2010 and indicates progress of completion. A surgery step 2012 may present information to the surgical staff in the operating room, such as a checklist or reminder. A surgical step may also require input to complete, such as but not limited to answering if the surgeon and surgical staff have worked together before.

The master data store 2014 persistently stores data describing some data that exists in the system for validating said data prior to persisting the data in the applicable system data stores.

The master CPT store 2016 persistently stores data describing master CPTs, or the type of surgery procedure(s) scheduled, performed or both. A surgeon 2002 is assigned CPTs the surgeon may perform and when assigned to a surgery 2010, those are the CPTs allowed as options for the surgery. A CPT is required when scheduling a surgery to build the surgery steps 2012 and estimated step duration for the surgery.

The scheduling module 2020 allows users to schedule a surgery case. The schedule module 2020 updates the surgery store 2010 and will be visible in the browser on the schedule for that organization, operating room and surgeon. Depending on user input the scheduling module may start, delete, or update the surgery case, such as time, or operating room.

The voice assistant module 2022 allows users to progress the surgery steps of a surgery case, notify a surgeon from the operating room, or receive information, such as but not limited to the surgeon is running late, inside the operating room or the like. Users can instruct the voice assistant module with voice commands that a surgery step has been completed, which will then be verified and updated by the scheduling module 2020.

The estimation algorithm, 2024, utilizes a machine learning predictive model, and/or any other artificial intelligence technique, to determine the estimated duration for each step in the perioperative process. In this embodiment the estimation algorithm 2024 uses data from multiple stores such as surgeon, patient and surgery to update the surgery steps with an estimated duration time by performing machine learning predictive modeling, and/or any other artificial intelligence technique resulting in an estimated surgery case duration by step and in total.

The EMR integration module 2026 parses data from the organization's medical records and can be used to fill stores such as surgeon 2002, patient 2004 and surgery 2010. Completed surgery data may be sent back to the organization's electronic medical record system with updated information.

The reporting module 2028 offers data from the various stores in compiled, searchable, sortable, filterable, and exportable ways. Graphics may be used to show data comparison to help assist user's understanding of the presented data.

The messaging module 2030 interfaces end users in the surgical tracker system 2000 with a messaging system. On any point in the system, a user may request and receive help via the messaging system.

The system administration module 2032 is used to create, update, view and or delete system related values and records such as operating rooms and voice assistant devices.

The user administration module 2034 is used to edit users such as surgeons and their basic profile information such as name, email, other contact info and the like.

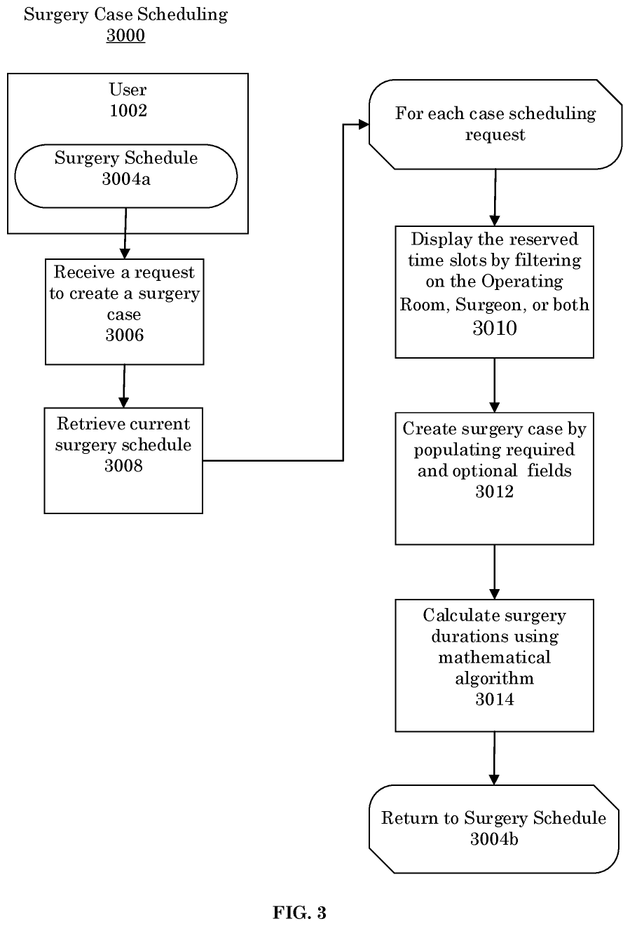

FIG. 3 shows a flowchart of a process for creating a surgery case information in the surgical tracking system, in accordance with an embodiment of the invention. The surgery case scheduling system 1000 user 1002 employs the surgery schedule user interface 3004a upon receipt of a request to create a surgery case 3006 to retrieve the current surgery schedule 3008. For each case scheduling request the surgical tracking system displays a calendar schedule 3010 of available date and time slots which may be filtered within an organization by operating room, surgeon or both. The user 1002 creates the surgery case by populating the user interface with required and optional data fields 3012. A process calculates the surgery duration using the estimation algorithm 2024. Upon user command the user is returned to the surgery schedule user interface 3004b.

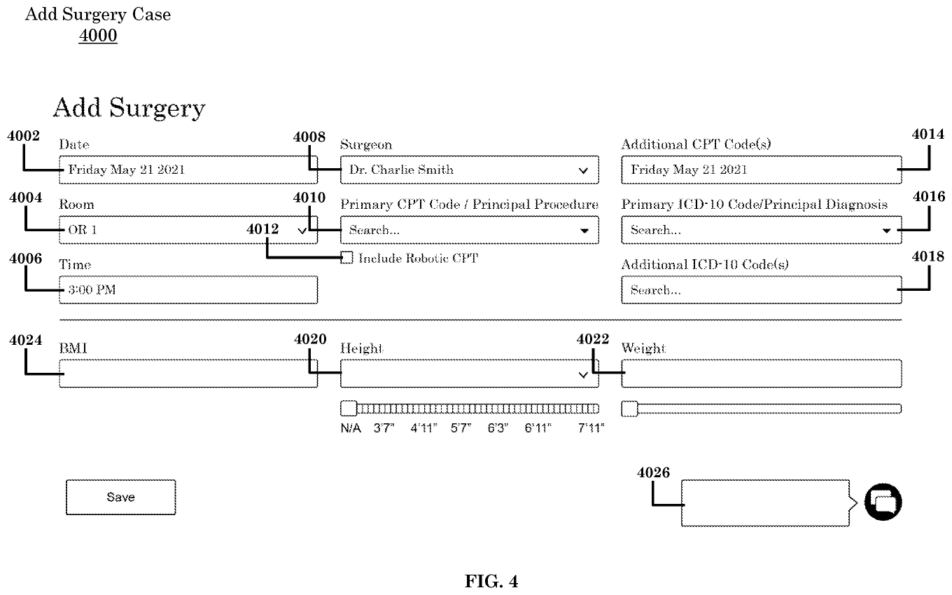

FIG. 4 shows an exemplary embodiment of the user interface for adding a surgery case 4000 which may contain date 4002 of the scheduled surgery, operating room 4004 where the surgery is to occur, the time 4006 of the scheduled surgery, surgeon name performing the surgery 4008, primary CPT code 4010, indicator of robotic surgery CPT code 4012, additional CPT codes 4014 primary ICD-10 code 4016, additional ICD-10 codes 4018, patient BMI 4024, patient height 4020, patient weight 4022, messaging window 4026 and the like.

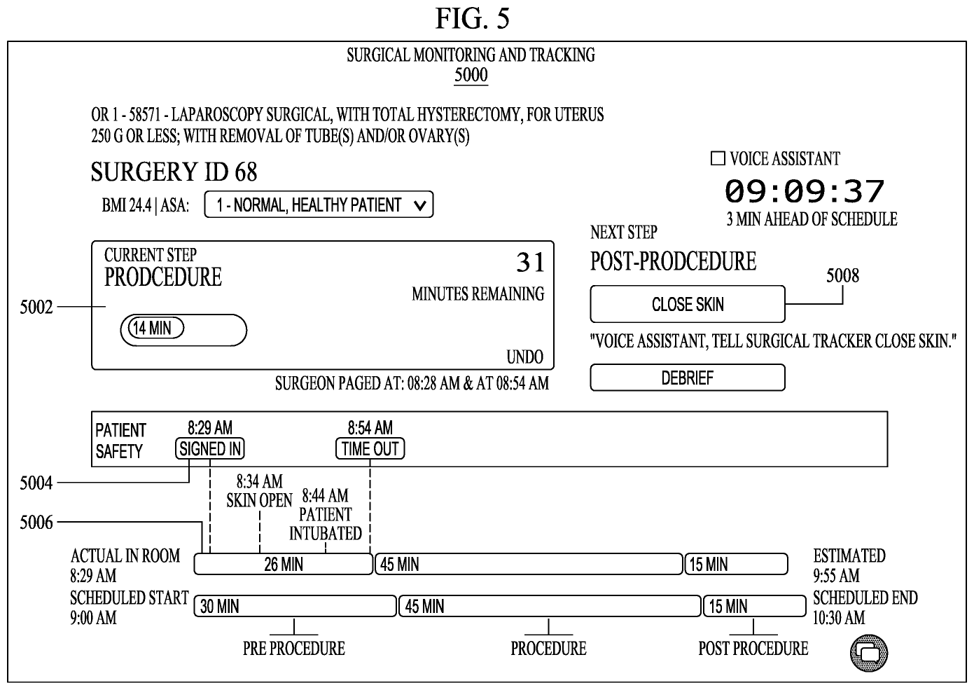

FIG. 5 shows an exemplary embodiment of the user interface for surgical monitoring and tracking 5000 which may contain timeline display of the status of the current surgical step 5002, timeline display of the patient safety surgical steps 5004, timeline display of the surgery actual and scheduled durations by step 5006, display of the next step 5008 and the like.

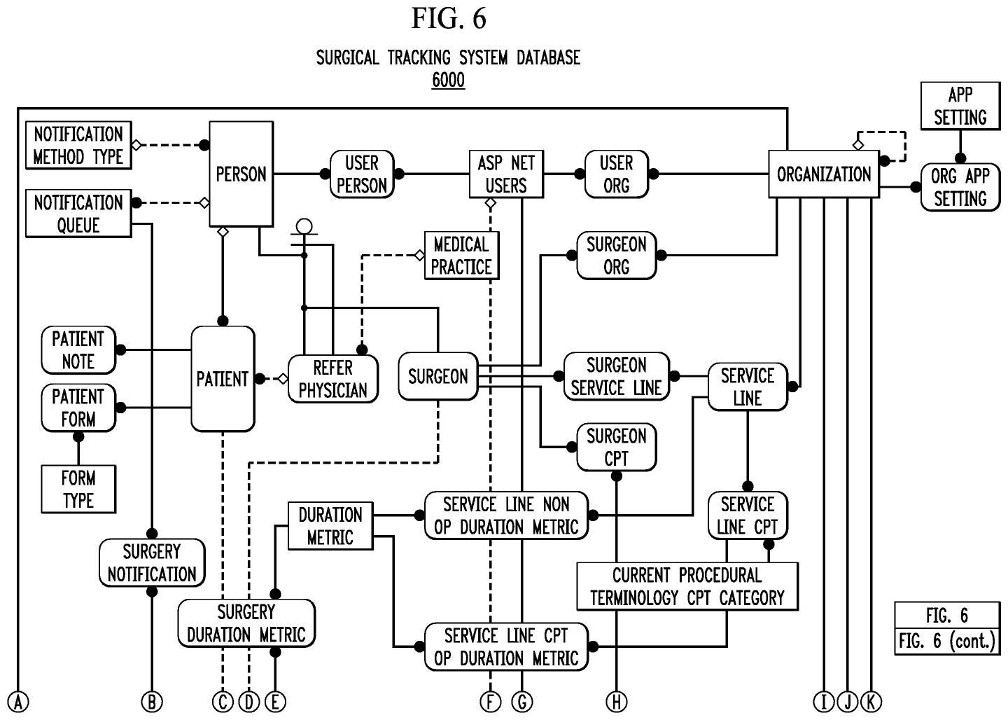

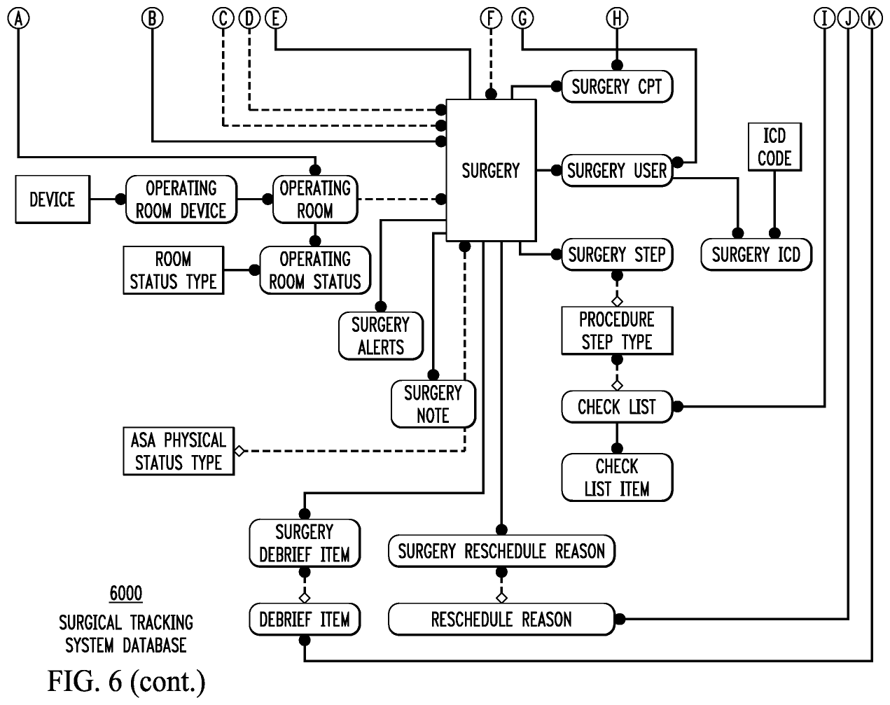

FIG. 6 shows an exemplary embodiment diagram of the surgical tracking system database 6000 of the persistent data stores used in accordance with one embodiment of the invention.

### CONCLUSION

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in Software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium or any type of media Suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1-8. (canceled)

9. A surgical tracking system comprising:
at least one processor; and
a memory configured to store program code, such that, when the program code is executed by the at least one processor, the at least one processor is configured to cause the system to:
receive, during a current surgical procedure comprising multiple steps having corresponding estimated durations, information comprising actual milestones and/or steps in the current surgical procedure;
calculate actual duration of each step in the current surgical procedure;
generate one or more displays during the current surgical procedure showing one or more comparisons between the estimated durations and the actual durations of the steps of the current surgical procedure;
generate, and store in the memory, an actual total duration of the current surgical procedure; and
generate, and store in the memory, an average total duration of the surgical procedure based on the actual total duration of the current surgical procedure and previously stored actual total durations of one or more previous surgical procedures.

10. The system of claim 9, wherein the system is configured to independently generate average total durations of surgical procedures taking into account at least one of:
different surgical procedures;
different surgeons;
different times of day;
different days of week;
different operating rooms;
different organizations; and
different patient characteristics.

11. The system of claim 9, wherein the system is further configured to generate, and store in the memory, average durations of the different surgical procedure steps based on the actual durations of the different steps in the current surgical procedure and previously stored actual durations of corresponding steps in one or more previous surgical procedures.

12. The system of claim 11, wherein the system is further configured to independently generate average durations of different surgical procedure steps taking into account at least one of:
different surgical procedures;
different surgeons;
different times of day;
different days of week;
different operating rooms;
different organizations; and
different patient characteristics.

13. The system of claim 9, wherein the system is configured to receive the information about the actual milestones electronically from a voice assistant device.

14. The system of claim 13, wherein the system is configured to control the voice assistant device to audibly describe a next step or milestone in the current surgical procedure.

15. The system of claim 9, wherein the system is configured to render the one or more displays on a terminal device electronically configured to the system.

16. The system of claim 9, wherein the system is configured to generate at least one display showing a next step in the current surgical procedure.

17. The system of claim 9, wherein the system is configured to generate at least one display showing how far the current surgical procedure is behind or ahead of schedule.

18. The system of claim 9, wherein the system is configured to generate at least one display showing (i) a first timeline comprising estimated durations of two or more steps in the current surgical procedure and (ii) a second timeline comprising actual durations of one or more steps already performed and estimated durations of one or more steps yet to be performed.

19. The system of claim 18, wherein the system is configured to generate the second timeline showing timing and identification of actual milestones during the current surgical procedure.

20. The system of claim 18, wherein the system is configured to generate:
the first timeline showing scheduled start and end times for the current surgical procedure; and
the second timeline showing actual start time and predicted end time for the current surgical procedure, where the predicted end time takes into account the actual durations of the one or more steps already performed.

21. The system of claim 18, wherein the system is configured to classify the current surgical procedure as standard or nonstandard based on comparison between the first timeline and the second timeline.

22. The system of claim 9, wherein the system is configured to generate at least one display showing one or more of:
identification of the current surgical procedure;
one or more patient characteristics;
current time of day; and
one or more times of day surgeon was electronically contacted.

23. The system of claim 9, wherein the system is configured to:
receive information about a future surgical procedure; and
provide a predicted total duration of the future surgical procedure based on the received information about the future surgical procedure and at least one average total duration stored in the memory.

24. The system of claim 23, wherein the received information about the future surgical procedure comprises one or more of:
characteristics of the future surgical procedure;
identification of surgeon;
identification of time of day;
identification of day of week;
identification of operating room;
identification of organization; and
identification of patient characteristics.

25. The system of claim 9, wherein the system is configured to correlate total actual duration of the current surgical procedure with patient outcome.

26. The system of claim 9, wherein the system is configured to generate one or more of the estimated durations of the steps based on average durations of the steps stored in the memory.

27. The system of claim 9, wherein the system is configured to generate one or more of the estimated durations of the steps based on information input by a user of the system.

28. The system of claim 9, wherein the system is configured to track a patient from admission to discharge.

29. The system of claim 9, wherein the system is configured to:
generate at least one display showing one or more patient safety checklists; and
receive a notification that a trigger event has occurred, the notification comprising a surgery event identifier and a checklist identifier capturing datetime the checklist was presented and acknowledged.

30. The system of claim 9, wherein the system is configured to exchange surgeon, patient, and/or surgery data with an organization's Electronic Medical Record (EMR) system using Health Level Seven (HL7) standard transactions.

31. The system of claim 9, wherein:
the system is configured to independently generate average total durations of surgical procedures taking into account at least one of:
different surgical procedures;
different surgeons;
different times of day;
different days of week;
different operating rooms;
different organizations; and
different patient characteristics;
the system is further configured to generate, and store in the memory, average durations of the different surgical procedure steps based on the actual durations of the different steps in the current surgical procedure and previously stored actual durations of corresponding steps in one or more previous surgical procedures;
the system is further configured to independently generate average durations of different surgical procedure steps taking into account at least one of:
different surgical procedures;
different surgeons;
different times of day;
different days of week;
different operating rooms;
different organizations; and
different patient characteristics;
the system is configured to receive the information about the actual milestones electronically from a voice assistant device;
the system is configured to control the voice assistant device to audibly describe a next step or milestone in the current surgical procedure;
the system is configured to render the one or more displays on a terminal device electronically configured to the system;
the system is configured to generate at least one display showing a next step in the current surgical procedure;
the system is configured to generate at least one display showing how far the current surgical procedure is behind or ahead of schedule;
the system is configured to generate at least one display showing (i) a first timeline comprising estimated durations of two or more steps in the current surgical procedure and (ii) a second timeline comprising actual durations of one or more steps already performed and estimated durations of one or more steps yet to be performed;
the system is configured to generate the second timeline showing timing and identification of actual milestones during the current surgical procedure;
the system is configured to generate:
the first timeline showing scheduled start and end times for the current surgical procedure; and
the second timeline showing actual start time and predicted end time for the current surgical procedure, where the predicted end time takes into account the actual durations of the one or more steps already performed;
the system is configured to classify the current surgical procedure as standard or nonstandard based on comparison between the first timeline and the second timeline;
the system is configured to generate at least one display showing one or more of:
identification of the current surgical procedure;
one or more patient characteristics;
current time of day; and
one or more times of day surgeon was electronically contacted;
the system is configured to:
receive information about a future surgical procedure; and
provide a predicted total duration of the future surgical procedure based on the received information about the future surgical procedure and at least one average total duration stored in the memory;
the received information about the future surgical procedure comprises one or more of:
characteristics of the future surgical procedure;
identification of surgeon;
identification of time of day;
identification of day of week;
identification of operating room;
identification of organization; and
identification of patient characteristics;
the system is configured to correlate total actual duration of the current surgical procedure with patient outcome;
the system is configured to generate one or more of the estimated durations of the steps based on average durations of the steps stored in the memory;
the system is configured to generate one or more of the estimated durations of the steps based on information input by a user of the system;
the system is configured to track a patient from admission to discharge;
the system is configured to:
generate at least one display showing one or more patient safety checklists; and
receive a notification that a trigger event has occurred, the notification comprising a surgery event identifier and a checklist identifier capturing datetime the checklist was presented and acknowledged; and
the system is configured to exchange surgeon, patient, and/or surgery data with an organization's Electronic Medical Record (EMR) system using Health Level Seven (HL7) standard transactions.

32. A method for a surgical tracking system, the method comprising the surgical tracking system:
receiving, during a current surgical procedure comprising multiple steps having corresponding estimated durations, information comprising actual milestones and/or steps in the current surgical procedure;
calculating actual duration of each step in the current surgical procedure;
generating one or more displays during the current surgical procedure showing one or more comparisons between the estimated durations and the actual durations of the steps of the current surgical procedure;
generating, and storing in memory, an actual total duration of the current surgical procedure; and
generating, and storing in the memory, an average total duration of the surgical procedure based on the actual total duration of the current surgical procedure and previously stored actual total durations of one or more previous surgical procedures.

33. A non-transitory storage medium comprising program code, wherein, when the program code is executed by at least one processor, the at least one processor is configured to cause a surgical tracking system to perform a method comprising the surgical tracking system:
receiving, during a current surgical procedure comprising multiple steps having corresponding estimated durations, information comprising actual milestones associated with actual start and actual end of each step in the current surgical procedure;
calculating actual duration of each step in the current surgical procedure;
generating one or more displays during the current surgical procedure showing one or more comparisons between the estimated durations and the actual durations of the steps of the current surgical procedure;
generating, and storing in memory, an actual total duration of the current surgical procedure; and
generating, and storing in the memory, an average total duration of the surgical procedure based on the actual total duration of the current surgical procedure and previously stored actual total durations of one or more previous surgical procedures.
